(19) United States
(12) Patent Application Publication
(10) Pub. No.: US 2007/0032505 A1
(43) Pub. Date: Feb. 8, 2007

(54) Crystalline form of gatifloxacin

(76) Inventors: Cosme Gomez, Torres de Alameda (ES); Javier Villasante Prieto, Madrid (ES); Francisco Palomo Nicolau, Alcala De Henares (ES)

Correspondence Address: WOLF BLOCK SCHORR AND SOLIS-COHEN LLP, 250 PARK AVENUE, NEW YORK, NY, 10177, US

(21) Appl. No.: 10/573,329

(22) PCT Filed: Nov. 5, 2004
(86) PCT No.: PCT/IB04/03652

§ 371 Date: Mar. 24, 2006

(30) Foreign Application Priority Data
Nov. 13, 2003 (ES) P-200302643

(51) Int. Cl. A61K 31/496; C07D 403/02

(52) U.S. Cl. 514/253.08; 544/363

(57) ABSTRACT

The present invention relates to a crystalline form of gatifloxacin obtainable by a process that comprises recrystallisation of the crude gatifloxacin in methanol and which is stable with a water content ranging between 2.5 and 4.5% by weight, to a process for preparing it and to the use thereof as an active substance in the preparation of pharmaceutical formulations.

### FIELD OF THE TECHNIQUE

The present invention relates to a new crystalline form of the active pharmaceutical substance gatifloxacin.

### PRIOR STATE OF THE ART

Gatifloxacin is the international common name of 1-cyclopropyl-6-fluoro-1,4-dihydro-ε-methoxy-7-(3-methyl-1-piperazinyl)-4-oxo-3-quinolinecarboxylic acid of formula (I), with application in medicine and known for its antibiotic activity:

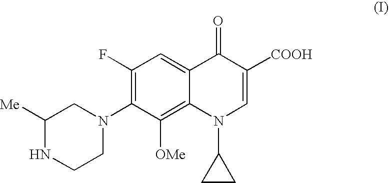

European patent application EP-A-230295 discloses the preparation of gatifloxacin, which is isolated in hemihydrate form (½ H₂O), corresponding to 2.34% in calculated weight of water.

European patent application EP-A-805156 discloses a sesquihydrated crystalline form ( 3/2 H₂O), corresponding to 6.72% in calculated weight of water.

Both crystalline forms have a tendency to absorb water and to form polymorphs with a higher content in hydration water.

Patent application WO-A-0222126 discloses gatifloxacin pentahydrate (5 H₂O), corresponding to 19.3% in calculated weight of water.

There therefore exists a need to have another hydrated crystalline form of gatifloxacin which is stable, having a lower water content.

The authors of this invention have discovered a new crystalline form of gatifloxacin, which they have designated form I, which is stable with a water content ranging between 2.5 and 4.5% by weight when it is in contact with the atmosphere at room temperature and with a relative humidity ranging between 20 and 70%.

### OBJECT OF THE INVENTION

The object of the present invention is a new crystalline form of gatifloxacin which is obtainable by means of a particular process.

Also object of this invention is the process for obtaining the new crystalline form of gatifloxacin.

Also forming part of the object of the present invention is the use of the new crystalline form of gatifloxacin for the manufacture of a medicament for the treatment of infectious diseases of bacterial origin.

### BRIEF DESCRIPTION OF THE DRAWINGS

FIG. 1 shows the powder X-ray diffractogram of the new crystalline form of gatifloxacin.

FIG. 2 shows the powder X-ray diffractogram of the gatifloxacin hemihydrate taken from North American patent U.S. Pat. No. 5,880,283. Said patent includes only the X-ray diffractogram, without the corresponding list of peaks shown at the different 2θ angles.

FIG. 3 shows the ¹³C nuclear magnetic resonance spectrum of the new crystalline form of gatifloxacin.

### DETAILED DESCRIPTION OF THE INVENTION

The authors of this invention have discovered a crystalline form of gatifloxacin, which they have designated form I, which is obtainable by means of a process comprising the following steps:
the crude gatifloxacin is dissolved in methanol by heating to reflux temperature, using between 50 and 65 volumes of methanol for each unit by weight of crude gatifloxacin,
it is cooled to a temperature between 15° C. and 25° C. within a period of time not exceeding 1.5 hours,
during the cooling process it is seeded with form I gatifloxacin,
it is then cooled to a temperature between 0° C. and 5° C. and kept at this temperature for at least 1 hour,
the solid product obtained is separated by filtration, and
the solid product is dried in an oven under vacuum to constant weight.

The crude gatifloxacin which is used as the starting product can be prepared as described in the Example of preparation set out below in this description, or according to the process described in Example 3 of the European patent application EP-A-230295.

The solution of crude gatifloxacin in methanol at reflux is prepared by using approximately 50 to 70 volumes of methanol for each unit by weight of crude gatifloxacin.

Once the crude gatifloxacin has been dissolved at the reflux temperature of the methanol, the solution is cooled to a temperature ranging between 15° C. and 25° C., which would be termed room temperature. In order to obtain the new crystalline form of gatifloxacin said cooling has to be carried out within a period of time not exceeding 1.5 hours.

During this period of cooling to room temperature seedings are carried out with form I gatifloxacin until a suspension containing an abundant precipitate is obtained.

The form I gatifloxacin which is used for seeding for the first time is prepared by means of the process described in the Example of preparation set out below in this description. On subsequent occasions the gatifloxacin obtained in Example 1 of this description can also be used.

The cooling of the suspension to a temperature between 0 and 5° C. is carried out by means of refrigeration with cold water and it is kept at this temperature for approximately one hour.

The solid obtained is separated by filtration and washed with cold methanol.

The moist solid is dried in an oven at approximately 40° C. in vacuo to constant weight.

The new crystalline form of gatifloxacin which is obtainable by this process has an initial water content ranging between 0.8 and 1.6% and stabilises with a water content ranging between 2.5 and 4.5% by weight, when it is in contact with the atmosphere at room temperature and with a relative humidity comprised between 20 and 70%, and it remains stable for at least 2 months in such conditions.

The new crystalline form of gatifloxacin usually stabilises in a period of time approximately equal to three days, but it could take longer to reach that degree of hydration if the degree of relative humidity was lower than 20%.

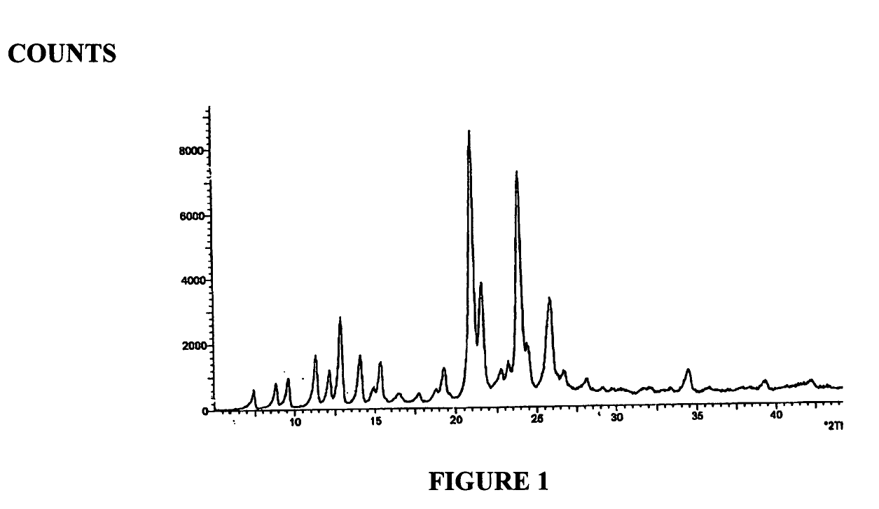

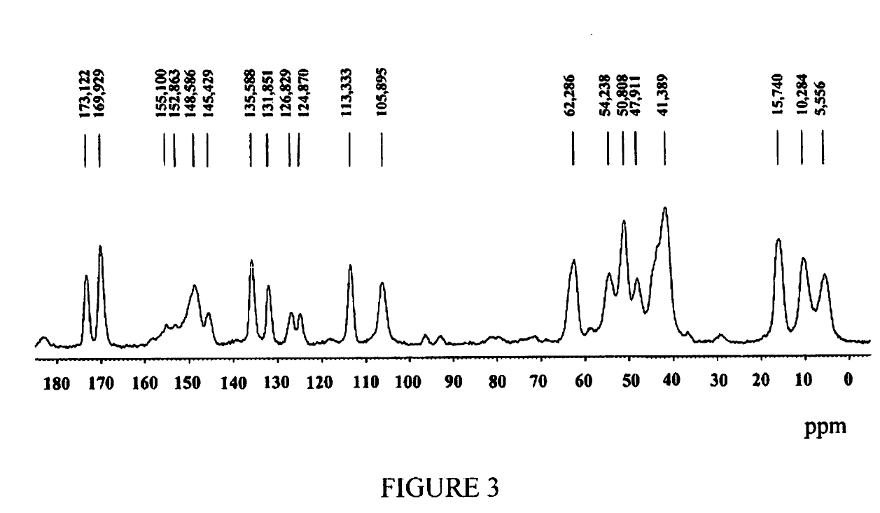

The crystalline form of gatifloxacin object of the present invention is characterized by its powder X-ray diffractogram (FIG. 1), ¹³C nuclear magnetic resonance spectrum (FIG. 3) and analysis of water content by the Karl-Fischer method.

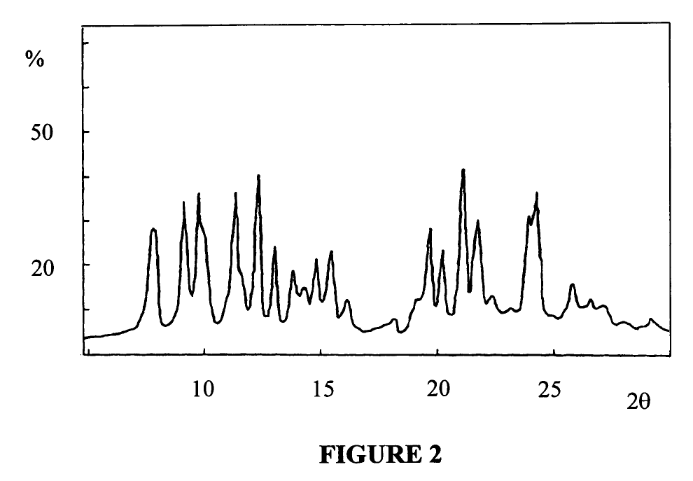

The X-ray diffractogram of the hemihydrate (FIG. 2) has been obtained from North American patent U.S. Pat. No. 5,880,283, in which the diffractograms of gatifloxacin hemihydrate (comparative substance) are compared with those of gatifloxacin sesquihydrate.

The form I gatifloxacin has an X-ray diffractogram which shows peaks at the 2θ angles 16.5±0.2 and 17.8±0.2 which are not present in the X-ray diffractogram of the gatifloxacin hemihydrate.

In its turn, the gatifloxacin hemihydrate has an X-ray diffractogram showing peaks at the 2θ angles 13.9±0.2, 14.5±0.2, 20.3±0.2, 22.5±0.2 and 24.2±0.2, which are not present in the X-ray diffractogram of the form I gatifloxacin object of the invention.

For recording the powder X-ray diffractograms a PHILIPS X'Pert automatic diffractometer equipped with a Cu tube and a graphite secondary monochromator of the following technical specifications was used:
Cu wavelength Kα: 1.5419 Å;
receiving slit: 0.1 mm;
Soller: 0.04 radians;
dispersion slit and divergence slit: 1°

The tube worked at 40 kV and 50 mA. Sweeping was carried out continuously in the 2θ interval between 5 and 40° with pass of 0.03° and 1-second pass time.

The ¹³C nuclear magnetic resonance spectrum (FIG. 3) was recorded on a solid sample of form I gatifloxacin.

Also object of the invention is the process for preparing the new crystalline form of gatifloxacin, which comprises the following steps:
the crude gatifloxacin is dissolved in methanol by means of heating to the reflux temperature,
it is cooled to a temperature ranging between 15° C. and 25° C. in a period of time slot exceeding 1.5 hours, and
it is seeded with form I gatifloxacin.

The solution of crude gatifloxacin in methanol at reflux is preferably prepared by using between 50 and 70 volumes of methanol for each unit by weight of crude gatifloxacin.

Once the crude gatifloxacin has been dissolved at the reflux temperature of methanol, the solution is cooled to room temperature, preferably to a temperature ranging between 15 and 25° C. To prepare the new crystalline form of gatifloxacin this cooling has to be carried out within a period of time not exceeding 1.5 hours.

During this period of cooling to room temperature, seedings with form I gatifloxacin are carried out until a suspension containing an abundant precipitate is obtained.

The form I gatifloxacin which is used for the seeding the first time is prepared by means of the process described in the Example of preparation set out below in this description. On subsequent occasions the form I gatifloxacin obtained in Example 1 of this description can also be used.

The process for preparing the new crystalline form of gatifloxacin further comprises the following steps:
the suspension is cooled to a temperature ranging between 0° C. and 5° C. and is kept at this temperature for at least 1 hour,
the solid product is filtered, and
the product is dried in an oven to constant weight.

The cooling of the suspension to a temperature between 0° C. and 5° C. is carried out by means of refrigeration with cold water and it is kept at this temperature for at least 1 hour.

The solid obtained is separated by filtration and washed with cold methanol.

The moist solid is dried in an oven, preferably at 40° C. in vacuo, to constant weight.

The new crystalline form of gatifloxacin, which is obtainable by this process, has an initial water content ranging between 0.8 and el 1.6%, and as being in contact with the atmosphere at room temperature and with a relative humidity ranging between 20 and 70%, it is stable with a water content between 2.5 and 4.5% by weight.

Surprisingly, it has been found that the new crystalline form of gatifloxacin containing between 2.5 and 4.5% of water by weight remains stable in its water content for at least 2 months, even at room temperature and with a relative humidity between 20 and 70%, and has excellent properties of disintegration and dissolution rate, which makes it very suitable for use as an active substance in pharmaceutical formulations, preferably for the manufacture of a medicament for the treatment of infectious diseases of bacterial origin.

The example which follows below is set out for the purposes of providing to the skilled man in the art a detailed explanation of a specific embodiment of the process for preparing the compound of the invention.

### EXAMPLE OF PREPARATION

### Preparing Form I Gatifloxacin for Seeding

10 g (0.0339 moles, 1 equivalent) of 1-cyclopropyl-6,7-difluoro-1,4-dihydro-8-methoxy-4-oxo-3-quinolinecarboxylic acid (CAS no.: 112811-72-0) is placed in a flask, 30 mL of acetonitryl (3 volumes) is added and the solution is heated to a temperature of 76-80° C. Once reflux has been attained, 3.28 g (0.0203 moles, 0.6 equivalents) of hexamethyldisilazane (HMDS) is added using a compensated addition funnel, maintaining the temperature. Once the addition is completed, the reaction is maintained with stirring for 1 hour at a temperature of 76-80° C.

Once this period has elapsed, the reaction mixture is cooled to a temperature between 0 and 15° C., and 5.78 g (0.0407 moles, 1.2 equivalents) of boron trifluoride ethyletherate is added, keeping the temperature below 15° C. Once the addition has finished, the temperature is allowed to rise to 15-25° C. and it is kept under these conditions for approximately 2 hours.

The pH of the mixture is then adjusted to an approximate value of 9 with triethylamine (approximately 2 mL). To the resulting suspension is added a solution of 10.19 g (0.1017 moles, 3 equivalents) of 2-methylpiperazine in 28 mL of acetonitryl, keeping the temperature between 15 and 25° C. The resulting amber solution is kept with stirring under these conditions for approximately 3 hours.

Once the reaction has been completed, the mixture is distilled at low pressure until a stirrable paste is obtained. At this point, 50 mL of methanol is added, the resulting suspension is raised to a temperature of 63-67° C. and kept under these conditions for approximately 5 hours. Once the reaction has been completed the mixture is cooled to a temperature of 25-35° C. over a water bath and then to a temperature of 0-5° C. over a water/ice bath for a further 1 hour. The resulting precipitate is filtered, washed with cold methanol (2×10 mL) and dried at 40° C. in an oven in vacuo to constant weight. 10.70 g of crude gatifloxacin is obtained, with a water content of 2.95% by weight. The yield of the process is 81.8%.

The crude product obtained is used for the seeding in Example 1.

The crude product is crystallised in methanol by dissolving 20 g of crude gatifloxacin in 1 l of methanol (50 volumes) at a temperature of 53-67° C. Once all the product has been dissolved it is placed to cool to a temperature of 30-40° C., and then to a temperature of 0-5° C. over a water/ice bath, maintaining it under these conditions for 1 hour. The resulting suspension is filtered and the solid retained is washed with 20 mL (1 volume) of cold methanol. The solid obtained is dried at 40° C. in a vacuum oven to obtain 18.65 g of gatifloxacin with a water content of 2.36% by weight.

The product obtained is used as crude gatifloxacin (starting product).

### Example 1

### Preparing Form I Gatifloxacin

To 39.23 g of crude gatifloxacin, prepared as in the Example of preparation, are added 2 l of methyl alcohol, and the suspension is heated to reflux temperature. Once a reflux regime has been reached, methyl alcohol is added until the product has totally dissolved. The total volume of methyl alcohol used is 2.69 l. Once the product has been dissolved the solution is cooled to room temperature in one hour, carrying out seedings with form I gatifloxacin (obtained in the Example of preparation) until the amount used in the seeding is inconsiderable compared with the amount of crystallised product. The resulting suspension is then cooled to a temperature between 0 and 5° C. over a water/ice bath, and is kept within that temperature range for 1 hour. Once the cooling period has elapsed, the solid is isolated by filtration and washed with cold methyl alcohol (2×40 mL). The product obtained is dried in a vacuum oven at 40° C. to constant weight.

31.25 g of a white solid is obtained, which has a water content, at the time of taking it out of the desiccator, of 1.5% by weight. The yield is 80.8%.

The product thus obtained is kept at room temperature in contact with the atmosphere, and three days later it has a water content of 3.22% by weight, which remains stable for at least 2 months at room temperature and with a relative humidity between 20 and 70%.

Table 1 shows the water—content values of the form I gatifloxacin obtained during the stability test:

TABLE 1

| | Time | | | | | | |
|---|---|---|---|---|---|---|---|
| | | 3 | 6 | | 13 | 17 | 68 |
| | 0 | hours | hours | 3 days | days | days | days |
| % water by | 1.5 | 2.2 | 2.3 | 3.2 | 2.9 | 3.0 | 3.3 |
| weight | | | | | | | |

The powder X-ray diffractogram recorded on the sample of form I gatifloxacin remains substantially unchanged over this entire period of time.

1. A crystalline form of gatifloxacin obtainable by means of a process comprising the steps of:
dissolving crude gatifloxacin in methanol by heating to reflux temperature, using between 50 and 65 volumes of methanol for each unit by weight of crude gatifloxacin,
cooling the methanol with dissolved gatifloxacin to a temperature ranging between 15° C. and 25° C. within a period of time not exceeding 1.5 hours,
during the cooling process it is seeded with form I gatifloxacin,
cooling the methanol with dissolved gatifloxacin to between 0° C. and 5° C. and keeping it at this temperature for at least 1 hour,
separating the solid product by filtration, and
drying the solid product in an oven under vacuum to constant weight.

2. A process for preparing a crystalline form of gatifloxacin, comprising the steps of:
dissolving crude gatifloxacin in methanol by means of heating to the reflux temperature,
cooling said methanol with dissolved gatifloxacin to a temperature between 15° C. and 25° C. in a period of time not exceeding 1.5 hours, and
seeding said methanol with dissolved gatifloxacin with form I gatifloxacin.

3. A process according to claim 2, wherein the crude gatifloxacin is dissolved in methanol, using between 50 and 70 volumes of methanol for each unit by weight of crude gatifloxacin.

4. A process according to claim 2, further comprising the steps of: claims 2 and 3, characterised in that it further comprises the following steps:
cooling the suspension to a temperature between 0° C. and 5° C. and kept at that temperature for at least 1 hour,
filtering the solid product and
drying the product in an oven to constant weight.

5. Use of the form of gatifloxacin according to claim 1 for the manufacture of a medicament for the treatment of infectious diseases of bacterial origin.
